(19) United States
(12) Patent Application Publication
(10) Pub. No.: US 2022/0175836 A1
(43) Pub. Date: Jun. 9, 2022

(54) PRIMING WITH TARGETED ACTIVATED T CELLS CAN ENHANCE CHEMO RESPONSIVENESS OF CANCER CELLS

(71) Applicant: University of Virginia Patent Foundation, Charlottesville, VA (US)

(72) Inventors: Lawrence G. Lum, Charlottesville, VA (US); Archana Thakur, Zion Crosseroads, VA (US)

(21) Appl. No.: 17/598,567

(22) PCT Filed: Mar. 27, 2020
(86) PCT No.: PCT/US2020/025461

§ 371 Date: Sep. 27, 2021

Related U.S. Application Data
(60) Provisional application No. 62/824,733, filed on Mar. 27, 2019.

(51) Int. Cl. A61K 35/17; A61K 45/06; A61P 35/00; A61K 39/00

(52) U.S. Cl. CPC A61K 35/17; A61K 45/06; A61K 2039/5158; A61K 39/001104; A61K 39/001106; A61P 35/00

(57) ABSTRACT

Methods and compositions for treating cancer in a subject in need thereof, comprising administering to the subject an effective amount of a targeted activated T cell, such as a bispecific antibody armed activated T cell (BAT), alone or in combination with an effective amount or a subtherapeutic amount of an additional therapeutic agent, wherein the targeted activated T cell selectively binds a cell of the cancer in the subject.

### CROSS REFERENCE TO RELATED APPLICATION

The presently disclosed subject matter claims the benefit of U.S. Provisional Patent Application Ser. No. 62/824,733, filed Mar. 27, 2019; the disclosure of which is incorporated herein by reference in its entirety.

### GOVERNMENT INTEREST

This invention was made with government support under Grant Nos. CA092344, CA140314, and CA182526, awarded by The National Institutes of Health. The government has certain rights in the invention.

### BACKGROUND

Pancreatic cancer (PC) has the worst survival rate of all cancers and five-year survival is less than 5% that has not improved over the last decade (1). Most patients are diagnosed at late stage with unresectable disease and up to 50% cases are diagnosed at metastatic stage that reduces their survival benefit from the conventional chemotherapy (2). Treatment of advanced or recurrent disease patients with gemcitabine in combination with nab-paclitaxel increased the median survival from 6.7 (Gem alone) to 8.7 months and with FOLFIRINOX (folinic acid, 5-FU, irinotecan, and oxaliplatin) the median survival increased from 6.8 to 11.2 months (3). Novel therapeutic strategies are needed to improve clinical results.

Multidrug resistance (both intrinsic and acquired) is thought to be a major reason for chemotherapeutic ineffectiveness of pancreatic cancer. The multidrug resistance (MDR) phenotype is mostly contributed by the members of the ATP-binding cassette (ABC) transporter superfamily and have been shown to be key mediators of drug efflux and drug resistance in many tumor types that decreases intracellular drug accumulation and its cytotoxic effects (4-6). Several transporters have been shown to contribute to MDR in vitro (7). P glycoprotein (P-gp)/ABCB1, ABCG2, ABCC1, ABCC2, and ABCC3 have been reported to be responsible for chemoresistance of PC (8). Another player to the refractory pancreatic cancer is the presence of CD44+/CD24+/EpCAM+ cancer stem like cells (CSC) that may contribute to the high recurrence rate after clinical remission. Improved and novel therapeutic strategies are needed for pancreatic cancer and for other cancers that develop drug resistance.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides a method for treating a cancer in a subject in need thereof. In some embodiments, the method comprises administering to the subject an effective amount of a composition comprising a targeted activated T Cell, such as a bispecific antibody (BiAb) armed activated T cell (BAT), which selectively binds a cell of the cancer in the subject, to thereby treat the cancer in the subject. In some embodiments, the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid or liquid tumor.

In some embodiments, the cancer is a drug resistant cancer or drug sensitive cancer. In some embodiments, the cancer is selected from the group comprising pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma.

In some embodiments, the method further comprises administering an effective amount or a subtherapeutic amount of an additional therapeutic agent contemporaneously with or after the administering of the targeted activated T cell. In some embodiments, the additional therapeutic agent is a chemotherapeutic agent or an anti-neoplastic agent. In some embodiments, the subtherapeutic amount of the additional therapeutic agent is a lower amount as compared to an amount of the additional therapeutic agent administered to a subject when the additional therapeutic agent is administered to a subject by itself.

In some embodiments, the presently disclosed subject matter provides a method for treating a cancer in a subject in need thereof, the method comprising administering to the subject an effective amount of a composition comprising a targeted activated T cell, such as a bispecific antibody (BiAb) armed activated T cell (BAT), which selectively binds a cell of the cancer in the subject; and administering an effective amount or a subtherapeutic amount of an additional therapeutic agent to the subject, to thereby treat the cancer in the subject. In some embodiments, the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid tumor.

In some embodiments, the cancer is a drug resistant cancer or drug sensitive cancer. In some embodiments, the cancer is selected from the group comprising pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma.

In some embodiments, the effective amount or the subtherapeutic amount of the additional therapeutic agent is administered contemporaneously with or after the administering of the targeted activated T cell. In some embodiments, the additional therapeutic agent is a chemotherapeutic agent or an anti-neoplastic agent. In some embodiments, the subtherapeutic amount of the additional therapeutic agent is a lower amount as compared to an amount of the additional therapeutic agent administered to a subject when the additional therapeutic agent is administered to a subject by itself.

In some embodiments, the BAT comprises anti-CD3 x anti-EGFR BiAb, anti-CD3 x anti-HER2 BiAb, anti-CD3 x anti-GD2 BiAb, anti-CD3 x anti-CD20 BiAb, or anti-CD3 x anti-SLAMF7 BiAb. In some embodiments, the BiAb used to arm the targeted activated T cell is a chemically heteroconjugated bispecific antibody or a recombinant bispecific antibody of any configuration. In some embodiments, the targeted activated T cells are produced from an apheresis product by anti-CD3 stimulation in the presence of IL-2, optionally at a range of about 20 to about 200 IU/ml, or wherein co-stimulated T cells are produced from an apheresis product by co-stimulation with anti-CD3/anti-CD28 coated beads, optionally in the presence of IL-2 at a range of about 20 to about 200 IU/ml, optionally at bead to cell ratios from about 1:3 to about 3:1.

In some embodiments, the subject is a mammalian subject. In some embodiments, the composition comprising the targeted activated T Cell and/or the additional therapeutic agent is/are adapted for administration for the treatment of a subject by intravenous administration, intrathecal injection, peritoneal injection, or direct injection into the tumor or surrounding tumor site.

In some embodiments, the presently disclosed subject matter provides a pharmaceutical composition comprising, consisting essentially of, or consisting of an effective amount of a targeted activated T cell, such as a bispecific antibody armed activated T cell (BAT), for use in treating a drug resistant cancer or a drug sensitive cancer in a subject in need thereof, wherein the targeted activated T cell selectively binds a cell of the cancer in the subject. In some embodiments, the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid tumor.

In some embodiments, the presently disclosed subject matter provides a pharmaceutical composition comprising, consisting essentially of, or consisting of an effective amount of a targeted activated T cell, such as an bispecific antibody armed activated T cell (BAT), for use in a method for treating a cancer in a subject in need thereof, in combination with an effective amount or a subtherapeutic amount of an additional therapeutic agent, wherein the targeted activated T cell selectively binds a cell of the cancer in the subject. In some embodiments, the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid tumor.

In some embodiments, the cancer is a drug resistant cancer or drug sensitive cancer. In some embodiments, the cancer is selected from the group comprising pancreatic cancer, breast cancer, prostate cancer, lung cancer, head and neck cancer, non-Hodgkin's lymphoma, acute myelogeneous leukemia, acute lymphoblastic leukemia, neuroblastoma, and glioblastoma.

In some embodiments, the effective amount or the subtherapeutic amount of the additional therapeutic agent is administered contemporaneously with or after the administering of the targeted activated T cell. In some embodiments, the additional therapeutic agent is a chemotherapeutic agent or an anti-neoplastic agent. In some embodiments, the subtherapeutic amount of the additional therapeutic agent is a lower amount as compared to an amount of the additional therapeutic agent administered to a subject when the additional therapeutic agent is administered to a subject by itself.

In some embodiments, the BAT comprises anti-CD3 x anti-EGFR BiAb, anti-CD3 x anti-HER2 BiAb, anti-CD3 x anti-GD2 BiAb, anti-CD3 x anti-CD20 BiAb, or anti-CD3 x anti-SLAMF7 BiAb. In some embodiments, the BiAb used to arm the targeted activated T cell is a chemically heteroconjugated bispecific antibody or a recombinant bispecific antibody of any configuration.

In some embodiments, the targeted activated T cells are produced from an apheresis product. In some embodiments, the targeted activated T cells are produced from an apheresis product by anti-CD3 stimulation in the presence of IL-2, optionally at a range of about 20 to about 200 IU/ml, or wherein co-stimulated T cells are produced from an apheresis product by co-stimulation with anti-CD3/anti-CD28 coated beads, optionally in the presence of IL-2 at a range of about 20 to about 200 IU/ml, optionally at bead to cell ratios from about 1:3 to about 3:1.

In some embodiments, the subject is a mammalian subject. In some embodiments, the composition comprising the targeted activated T cell and/or the additional therapeutic agent is/are adapted for administration for the treatment of a subject by intravenous administration, intrathecal injection, peritoneal injection, or direct injection into the tumor or surrounding tumor site.

Accordingly, it is an object of the presently disclosed subject matter to provide compositions and methods for treating cancer. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIG. 1 is a series of graphs showing the cytotoxicity by unarmed activated T cells (ATC) (top) EGFR BATs (lower left) and HER2 BATs (lower right) by ⁵¹Cr release assay against parental, gemcitabine-resistant (GEM) and cisplatin resistant (CIS) derivatives of MiaPaCa-2 pancreatic cancer cell line.

FIG. 2A is a graph showing cytotoxicity of PANC-1 cells pretreated with BATs followed by treatment with a concentration that is 4 or 2 times lower than the IC₅₀ concentration of cisplatin (CIS). The IC₅₀ concentration for PANC-1 cells is 14.0 μM. Cytotoxicity increased in BATs primed target cells compared to non-BATs primed cells.

FIG. 2B is a graph showing cytotoxicity of MiaPaCa-2 cells pretreated with BATs followed by treatment with a concentration that is 5 or 2.5 times lower than the IC₅₀ concentration of cisplatin (CIS). The IC₅₀ concentration for MiaPaCa-2 cells is 18.6 μM. Cytotoxicity increased in BATs primed target cells compared to non-BATs primed cells.

FIG. 2C is a graph showing cytotoxicity for BxPC-3 cells pretreated with BATs followed by treatment with a concentration that is 5 or 2.5 times lower than the IC₅₀ dose of cisplatin (CIS). The IC₅₀ concentration for BxPC-3 cells is 6.9 μM. Cytotoxicity increased in BATs primed target cells compared to non-BATs primed cells.

FIG. 2D is a graph showing cytotoxicity for CFPAC cells pretreated with BATs followed by treatment with a concentration that is 5 or 2.5 times lower than the IC₅₀ concentration of cisplatin (CIS). The IC₅₀ concentration for CFPAC cells is 10.0 μM. Cytotoxicity increased in BATs primed target cells compared to non-BATs primed cells.

FIG. 2E is a graph showing cytotoxicity for BxPC-3 cells pretreated with BATs followed by treatment with a concentration that is 5 or 2.5 times lower than the IC₅₀ dose of SFU. The IC₅₀ concentration for BxPC-3 cells is 14.6 μM. Cytotoxicity increased in BATs primed target cells compared to non-BATs primed cells.

FIG. 2F is a graph showing cytotoxicity for CFPAC cells pretreated with BATs followed by treatment with 5 or 2.5 times lower concentrations of the IC₅₀ dose of SFU. The IC₅₀ dose for CFPAC cells is 5 Cytotoxicity increased in BATs primed target cells compared to non-BATs primed cells.

FIG. 3 is a graph showing the cytotoxicity of cisplatin (CIS) in cisplatin-resistant PANC-1 cells pretreated with BATs followed by treatment with the IC₅₀ concentration of CIS and concentrations that are 5 or 2.5 times lower than of the IC₅₀ concentration.

FIG. 4 is a schematic drawing showing (top) the preparation of an EGFR bispecific antibody (EGFRBi) via chemical conjugation of anti-CD3 to anti-EGFR; (bottom left) the arming of activated T cells (ATCs) with the EGFR bispecific antibody (EGFRBi) to provide EGFR BATs; and (bottom right) targeted killing using EGFR BATs, which, upon engagement of a tumor cell, release an array of cytokines, which leads to the destruction of the tumor target and stimulation of the endogenous immune system.

FIGS. 5A and 5B: ⁵¹Cr release was used to test whether BATs at E:T 25:1 can be used to target then retarget parental and drug-resistant L3.6. FIG. 5A is a graph showing cytotoxicity in parental and drug-resistant L3.6 cells pretreated with ATC or aATC followed by retreatment with EGFR BATs. FIG. 5B is a graph showing cytotoxicity in parental and drug resistant L3.6 cells pretreated with ATC or aATC followed by retreatment with HER2 BATs. These data suggest that BATs can target and kill parental and drug-resistant pancreatic tumor cell lines.

FIG. 6 is a graph showing the xCelligence RTCA system evaluation of EGFR BATs sensitization of parental pancreatic cancer cell lines to chemotherapy. The vertical black line denotes the normalization point. The arrow on the left indicates when ATC or EGFR BATs were added, and the arrow on the right indicates when T cells were washed and CIS was introduced. Conditions were run in triplicate. Dashed lines depict standard deviation. IC₅₀ CIS: MiaPaCa-2=18.6 μM; PANC-1=14.0 μM. MiaPaCa-2 primed with EGFR BATs at E:T of 2:1 show enhanced sensitivity to CIS at half the IC₅₀ concentration over the unprimed control.

FIG. 7 is a graph showing the xCelligence RTCA system evaluation of parental MiaPaCa-2 primed with EGFR BATs at various E:T ratios (1:1, 1:2, and 1:4) and then treated with CIS at the IC₅₀ concentration. The vertical black line denotes the normalization point. The arrow on the left indicates when EGFR BATs were added, and the arrow on the right indicates when EGFR BATs were washed and CIS was introduced. Dashed lines depict standard deviation. Conditions were run in triplicate. IC₅₀ CIS: MiaPaCa=18.6 μM. EGFR BATs priming at low E:T continually increased sensitivity to the cytotoxic effects of CIS at the IC₅₀ concentration.

FIGS. 8A and 8B are a two series of graphs showing cytotoxicity measured using xCelligence to test whether exposure of parental MiaPaCa-2 (FIG. 8A, n=4) or parental PANC-1 (FIG. 8B, n=3) to EGFR BATs at a 2:1 E:T increases CIS sensitivity when treated with 25%, 50%, or 100% the IC₅₀ concentration of CIS. Conditions were run in triplicate. Cell index (CI) readings from the xCelligence were taken 72 HR post chemotherapy, normalized, and converted to % Cytotoxicity using the following formula: % Cytotoxicity=(CI tumor only−CI treated)/(CI tumor only)×100. IC₅₀ CIS: MiaPaCa-2=18.6 μM; PANC-1=14.0 μM. Paired t-test, *P<0.05. Cytotoxic effects of CIS were generally greater after priming when compared to CIS alone at and below IC₅₀ concentrations.

FIG. 9 is a series of graphs showing cytotoxicity measured using xCelligence in CIS-resistant MiaPaCa-2 primed with EGFR BATs at an E:T of 1:2 before exposure to various concentrations of CIS. Cell index (CI) readings from the xCelligence were taken 72

HR post chemotherapy, normalized, and converted to % Cytotoxicity using the following formula: % Cytotoxicity=(CI tumor only−CI treated)/(CI tumor only)×100. IC₅₀ CIS: MiaPaCa=18.6 μM. Conditions were run in triplicate. Cytotoxic effects of CIS appeared to be greater after priming when compared to CIS alone.

### DETAILED DESCRIPTION

Headings are included herein for reference and to aid in locating certain sections. These headings are not intended to limit the scope of the concepts described therein under, and these concepts can have applicability in other sections throughout the entire specification.

Treatment of unresectable pancreatic tumor is significantly influenced by resistance of cancer cells to anti-cancer drugs. The multidrug resistance (MDR) phenotype is mostly contributed by the members of the ATP-binding cassette (ABC) transporter superfamily and have been shown to be key mediators of drug efflux and drug resistance in many tumor types including the refractory pancreatic cancer and CD44+/CD24+/EpCAM+ cancer stem like cells (CSC) that contribute to the high recurrence rate after clinical remission.

In accordance with aspects of the presently disclosed subject matter, data from a clinical study in metastatic pancreatic cancer (MPC) patients suggest development of dramatic responses to the same chemotherapy after multiple infusions of bispecific antibody (Anti-CD3 x Anti-EGFR) armed activated T cells (EGFR BATs) that patient received prior to EGFR BATs infusions. Thus, aspects of the presently disclosed subject matter evaluate a) whether BATs can target drug resistant pancreatic cancer cells; b) whether priming of drug sensitive or drug resistant pancreatic cancer cell lines with BATs can sensitize these cells for enhanced responsiveness to chemotherapeutic drugs.

The presently disclosed data in drug resistant cell lines show increased proportion of CD44+/CD24+/EpCAM+ cancer stem like cells as well as an increased number of ABC transporter ABCG2 positive cells compared to the parental cell lines. EGFR BATs (n=6) were as effective in targeting gemcitabine (GEM) or cisplatin (CIS) resistant MiaPaCa-2 (Median cytotoxicity was 48% compared to 33% of parental cells) and L3.6 (Median cytotoxicity was 44% compared to 30% of parental cells) cell lines as parental cell lines, produced Th1 cytokines, IFN-gamma and TNF-alpha, and chemokines, MIP-1b and RANTES. Priming of drug sensitive or drug resistant cells with EGFR BATs followed by retargeting with 3-5× lower IC₅₀ levels of CIS and GEM showed enhanced cytotoxicity. These data suggest that BATs mediated killing of chemoresistant and chemosensitive tumor cells and release of Th1 cytokines modulate the tumor microenvironment to enhance anti-tumor immune responses and sensitize tumor for enhanced chemotherapeutic responsiveness.

### DEFINITIONS

In describing and claiming the presently disclosed subject matter, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, in some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

As used herein, the phrase “biological sample” refers to a sample isolated from a subject (e.g., a biopsy, blood, serum, etc.) or from a cell or tissue from a subject (e.g., RNA and/or DNA and/or a protein or polypeptide isolated therefrom). Biological samples can be of any biological tissue or fluid or cells from any organism as well as cells cultured in vitro, such as cell lines and tissue culture cells. Frequently the sample will be a “clinical sample” which is a sample derived from a subject (i.e., a subject undergoing a diagnostic procedure and/or a treatment). Typical clinical samples include, but are not limited to cerebrospinal fluid, serum, plasma, blood, saliva, skin, muscle, olfactory tissue, lacrimal fluid, synovial fluid, nail tissue, hair, feces, urine, a tissue or cell type, and combinations thereof, tissue or fine needle biopsy samples, and cells therefrom. Biological samples can also include sections of tissues, such as frozen sections or formalin fixed sections taken for histological purposes.

As used herein, term “comprising”, which is synonymous with “including,” “containing”, or “characterized by”, is inclusive or open-ended and does not exclude additional, unrecited elements and/or method steps. “Comprising” is a term of art used in claim language which means that the named elements are present, but other elements can be added and still form a composition or method within the scope of the presently disclosed subject matter. By way of example and not limitation, a pharmaceutical composition comprising a particular active agent and a pharmaceutically acceptable carrier can also contain other components including, but not limited to other active agents, other carriers and excipients, and any other molecule that might be appropriate for inclusion in the pharmaceutical composition without any limitation.

As used herein, the phrase “consisting of” excludes any element, step, or ingredient that is not particularly recited in the claim. When the phrase “consists of” appears in a clause of the body of a claim, rather than immediately following the preamble, it limits only the element set forth in that clause; other elements are not excluded from the claim as a whole. By way of example and not limitation, a pharmaceutical composition consisting of an active agent and a pharmaceutically acceptable carrier contains no other components besides the particular active agent and the pharmaceutically acceptable carrier. It is understood that any molecule that is below a reasonable level of detection is considered to be absent.

As used herein, the phrase “consisting essentially of ” limits the scope of a claim to the specified materials or steps, plus those that do not materially affect the basic and novel characteristic(s) of the claimed subject matter. By way of example and not limitation, a pharmaceutical composition consisting essentially of an active agent and a pharmaceutically acceptable carrier contains active agent and the pharmaceutically acceptable carrier, but can also include any additional elements that might be present but that do not materially affect the biological functions of the composition in vitro or in vivo.

With respect to the terms “comprising”, “consisting essentially of”, and “consisting of”, where one of these three terms is used herein, the presently disclosed and claimed subject matter encompasses the use of either of the other two terms. For example, “comprising” is a transitional term that is broader than both “consisting essentially of” and “consisting of”, and thus the term “comprising” implicitly encompasses both “consisting essentially of” and “consisting of”. Likewise, the transitional phrase “consisting essentially of” is broader than “consisting of”, and thus the phrase “consisting essentially of” implicitly encompasses “consisting of”.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Ayes (birds), and Mammalia (mammals)), and all Orders and Families encompassed therein. In some embodiments, a subject is a human.

Similarly, all genes, gene names, gene products, and other products disclosed herein are intended to correspond to orthologs or other similar products from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, any genes specifically mentioned herein and for which Accession Nos. for various exemplary gene products disclosed in the GENBANK® biosequence database, are intended to encompass homologous and variant genes and gene products from humans and other animals including, but not limited to other mammals.

The methods of the presently disclosed subject matter are particularly useful for warm-blooded vertebrates. Thus, the presently disclosed subject matter concerns mammals and birds. More particularly contemplated is the isolation, manipulation, and use of stem cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also contemplated is the isolation, manipulation, and use of stem cells from livestock, including but not limited to domesticated swine (pigs and hogs), ruminants, horses, poultry, and the like.

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 9%, in some embodiments no more than 8%, in some embodiments no more than 7%, in some embodiments no more than 6%, in some embodiments no more than 5%, in some embodiments no more than 4%, in some embodiments no more than 3%, in some embodiments no more than 2%, in some embodiments no more than 1%, and in some embodiments no more than 0% of the components of a collection of entities does not have a given characteristic.

The terms “additional therapeutically active compound” or “additional therapeutic agent”, as used in the context of the presently disclosed subject matter, refer to the use or administration of a compound for an additional therapeutic use for a particular injury, disease, or disorder being treated. Such a compound, for example, could include one being used to treat an unrelated disease or disorder, or a disease or disorder which is not responsive to the primary treatment for the injury, disease or disorder being treated. Diseases and disorders being treated by the additional therapeutically active agent include, for example, cancer. The additional compounds can also be used to treat symptoms associated with the injury, disease, or disorder, including, but not limited to, pain and inflammation.

The term “adult” as used herein, is meant to refer to any non-embryonic or non-juvenile subject.

As used herein, an “agonist” is a composition of matter which, when administered to a mammal such as a human, enhances or extends a biological activity attributable to the level or presence of a target compound or molecule of interest in the subject.

A disease or disorder is “alleviated” if the severity of a symptom of the disease, condition, or disorder, or the frequency with which such a symptom is experienced by a subject, or both, are reduced.

“Allogeneic” refers to cells or to a graft derived from a different animal of the same species.

As used herein, amino acids are represented by the full name thereof, by the three letter code corresponding thereto, or by the one-letter code corresponding thereto, as indicated in Table 1:

TABLE 1
Amino Acid Codes and Functionally Equivalent Codons

| | 3- | 1- | |
|---|---|---|---|
| | Letter | Letter | |
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter. The term “amino acid” is used interchangeably with “amino acid residue,” and can refer to a free amino acid or to an amino acid residue of a peptide. It will be apparent from the context in which the term is used whether it refers to a free amino acid or a residue of a peptide.

Amino acids can be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

Amino acids have the following general structure:

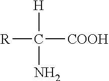

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino-and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter which when administered to a mammal such as a human, inhibits a biological activity attributable to the level or presence of a compound or molecule of interest in the subject.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter can exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ can be reduced under mild conditions to break the disulfide linkage in the hinge region, thereby converting the F(ab′)₂ dimer into an Fab₁ monomer. The Fab₁ monomer is essentially a Fab with part of the hinge region (see Paul, 1993). While various antibody fragments are defined in terms of the digestion of an intact antibody, one of skill will appreciate that such fragments can be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all intact antibody molecules.

An “antibody light chain”, as used herein, refers to the smaller of the two types of polypeptide chains present in all intact antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response can involve either antibody production, or the activation of specific immunologically-competent cells, or both. An antigen can be derived from organisms, subunits of proteins/antigens, killed or inactivated whole cells or lysates.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence can be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

The term “autologous”, as used herein, refers to something (e.g., a cell or cells) that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, AR8, etc., to which other ingredients can be added. The term does not exclude media which have been prepared or are intended for specific uses, but which upon modification can be used for other cell types, etc.

The term “biocompatible”, as used herein, refers to a material that does not elicit a substantial detrimental response in the host.

The term “biodegradable”, as used herein, means capable of being biologically decomposed. A biodegradable material differs from a non-biodegradable material in that a biodegradable material can be biologically decomposed into units which can be either removed from the biological system and/or chemically incorporated into the biological system.

The term “biological sample”, as used herein, refers to samples obtained from a living organism, including skin, hair, tissue, blood, plasma, cells, sweat, and urine.

The term “bioresorbable”, as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate can, for example, be redeposited as bone mineral, or by being otherwise re-utilized within the body, or excreted. “Strongly bioresorbable”, as the term is used herein, means that at least 80% of the total mass of material implanted is resorbed within one year.

A “bispecific antibody,” as used herein, refers to an antibody having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the coexpression of two immunoglobulin heavy chain/light chain pairs. See, e.g., Milstein et al. (1983) Nature 305: 537-39. Alternatively, bispecific antibodies can be prepared using chemical linkage. See, e.g., Brennan et al. (1985) Science 229:81. Bispecific antibodies include bispecific antibody fragments. See, e.g., Bolliger et al. (1993) Proc. Natl. Acad. Sci. U.S.A. 90:6444-48, Gruber et al. (1994) J. Immunol. 152:5368.

The phrases “cell culture medium”, “culture medium” (plural “media” in each case), and “medium formulation” refer to a nutritive solution for cultivating cells and may be used interchangeably.

A “conditioned medium” is one prepared by culturing a first population of cells or tissue in a medium, and then harvesting the medium. The conditioned medium (along with anything secreted into the medium by the cells) can then be used in any desired way, such as to treat a disease or disorder in a subject, or to support the growth or differentiation of a second population of cells.

As used herein, the term “conservative amino acid substitution” is defined herein as an amino acid exchange within one of the five groups summarized in the following Table 2.

TABLE 2
Conservative Amino Acid Substitutions

| Group | Characteristics | Amino Acids |
|---|---|---|
| A. | Small aliphatic, nonpolar or slightly | Ala, Ser, Thr, Pro, |
| | polar residues | Gly |
| B. | Polar, negatively charged residues and | Asp, Asn, Glu, Gln |
| | their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “control” cell, tissue, sample, or subject is a cell, tissue, sample, or subject of the same type as a test cell, tissue, sample, or subject. The control can, for example, be examined at precisely or nearly the same time the test cell, tissue, sample, or subject is examined. The control can also, for example, be examined at a time distant from the time at which the test cell, tissue, sample, or subject is examined, and the results of the examination of the control can be recorded so that the recorded results can be compared with results obtained by examination of a test cell, tissue, sample, or subject. The control can also be obtained from another source or similar source other than the test group or a test subject, where the test sample is obtained from a subject suspected of having a disease or disorder for which the test is being performed.

A “test” cell, tissue, sample, or subject is one being examined or treated.

A tissue “normally comprises” a cell if one or more of the cells are present in the tissue in an animal not afflicted with a disease or disorder.

A “compound”, as used herein, refers to any type of substance or agent that is commonly considered a drug, or a candidate for use as a drug, combinations, and mixtures of the above, as well as polypeptides and antibodies of the presently disclosed subject matter.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

“Chemokine”, as used herein, refers to an intercellular signaling molecule involved in the chemotaxis of white blood cells, such as T cells.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that can be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect. A “therapeutically effective amount” means an effective amount of an agent being used in treating or preventing a disease or disorder.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length.

As used herein, a “functional” molecule is a molecule in a form in which it exhibits a property or activity by which it is characterized.

As used herein, a “functional biological molecule” is a biological molecule in a form in which it exhibits a property by which it is characterized. A functional enzyme, for example, is one which exhibits the characteristic catalytic activity by which the enzyme is characterized.

The term “growth factor” as used herein means a bioactive molecule that promotes the proliferation of a cell or tissue. Growth factors useful in the presently disclosed subject matter include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9, and 10, nerve growth factors (NGF) including NGF 2.5s, NGF 7.0s, and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul (1990) Methods for assessing the statistical significance of molecular sequence features by using general scoring schemes. Proc Natl Acad Sci U S A 87:2264-2268, modified as in Karlin & Altschul (1993) Applications and statistics for multiple high-scoring segments in molecular sequences. Proc Natl Acad Sci U S A 90:5873-5877). This algorithm is incorporated into the NBLAST and XBLAST programs (see Altschul et al. (1990a) Basic local alignment search tool. J Mol Biol 215:403-410; Altschul et al. (1990b) Protein database searches for multiple alignments. Proc Natl Acad Sci U S A 87:14:5509-5513, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997) Gapped BLAST and PSI- BLAST: a new generation of protein database search programs. Nucleic Acids Res 25:3389-3402. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block”.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “injecting or applying” includes administration of a compound or composition of the presently disclosed subject matter by any number of routes and approaches including, but not limited to, topical, oral, buccal, intravenous, intratumoral, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the composition of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together with a container, which contains the identified compound. Alternatively, the instructional material can be shipped separately from the container with the intention that the instructional material and the compound be used cooperatively by the recipient.

Used interchangeably herein are the terms “isolate” and “select”.

The terms “isolate”, “isolated”, “isolating”, and grammatical variations thereof when used in reference to compositions or cells, refers to a single composition or cell of interest, or a population of compositions or cells of interest, at least partially isolated from other cell types or other cellular material with which it occurs in a culture or a tissue of origin.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

A “receptor” is a compound that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule or bivalent group derived therefrom that joins two other molecules covalently or noncovalently, e.g., through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

Micro-RNAs are generally about 16-25 nucleotides in length. In some embodiments, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′ -UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intratumoral, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human).

Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide can serve a non-coding function (e.g., promoter, origin of replication, ribosome-binding site, etc.), as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which can be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

A “significant detectable level” is an amount of contaminate that would be visible in the presented data and would need to be addressed/explained during analysis of the forensic evidence.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases, scFv are expressed on the surface of an engineered cell, for the purpose of selecting particular scFv that bind to an antigen of interest.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By the term “specifically binds”, as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not always limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest.

A “subject” of diagnosis or treatment is an animal, including a human. It also includes pets and livestock.

As used herein, a “subject in need thereof” is a patient, animal, mammal, or human, who will benefit from a method or compositions of the presently disclosed subject matter.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference sequence. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; in some embodiments in 7% (SDS), 0.5 M NaPO4, 1 mM EDTA at 50° C. with washing in 1×SSC, 0.1% SDS at 50° C.; in some embodiments 7% SDS, 0.5 M NaPO4, 1 mM EDTA at 50° C. with washing in 0.5×SSC, 0.1% SDS at 50° C.; and more in some embodiments in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package, and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, molecule, or the like, which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., those disclosed in the EXAMPLES, or in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cell united perform a specific function; (2) a part of an organism consisting of an aggregate of cells having a similar structure and function; or (3) a grouping of cells that are similarly characterized by their structure and function, such as muscle or nerve tissue.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

The term “transfection” is used interchangeably with the terms “gene transfer”, “transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The terminology used herein is for the purpose of describing the particular versions or embodiments only, and is not intended to limit the scope of the presently disclosed subject matter. All publications mentioned herein are incorporated by reference in their entirety.

### II. Representative Embodiments

### II.A. General Considerations

Treatment of unresectable pancreatic tumor is significantly influenced by resistance of cancer cells to anti-cancer drugs. The multidrug resistance (MDR) phenotype is mostly contributed by the members of the ATP-binding cassette (ABC) transporter superfamily and have been shown to be key mediators of drug efflux and drug resistance in many tumor types including the refractory pancreatic cancer and CD44+/CD24+/EpCAM+ cancer stem like cells (CSC) that contribute to the high recurrence rate after clinical remission. The presently disclosed subject matter relates in some embodiments, to the use of activated T cells, such as bispecific antibody armed activated T cells (BATs), to target drug resistant and drug sensitive cancer cells, such as but not limited to pancreatic cancer cells and to the use of activated T cells, such as bispecific antibody armed activated T cells (BATs), can sensitize these cells for enhanced responsiveness to chemotherapeutic drugs.

Aspects of the presently disclosed subject matter involve data in drug resistant cell lines showing an increased proportion of CD44+/CD24+/EpCAM+ cancer stem like cells as well as an increased number of ABC transporter ABCG2 positive cells as compared to the parental cell lines. EGFR BATs (n=6) were as effective in targeting gemcitabine (GEM) or cisplatin (CIS) resistant MiaPaCa-2 (Median cytotoxicity was 48% compared to 33% of parental cells) and L3.6 (Median cytotoxicity was 44% compared to 30% of parental cells) cell lines as parental cell lines, produced Th1 cytokines, IFN-γ and TNF-α, and chemokines, MIP-1b and RANTES. Priming of drug sensitive or drug resistant cells with EGFR BATs followed by retargeting with 3-5× lower IC₅₀ levels of CIS and GEM showed enhanced cytotoxicity. These data show that BATs mediated killing of chemoresistant tumor cells and release of Th1 cytokines modulate the tumor microenvironment to enhance anti-tumor immune responses and sensitize tumor for enhanced chemotherapeutic responsiveness.

### II.B. Representative Treatment Methods and Compositions

In some embodiments, the presently disclosed subject matter provides a method for treating a cancer in a subject in need thereof. In some embodiments, the method comprises administering to the subject an effective amount of a composition comprising a targeted activated T cell, such as a bispecific antibody (BiAb) armed activated T cell (BAT) which selectively binds a cell of the cancer in the subject, to thereby treat the cancer in the subject. In some embodiments, the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid or liquid tumor.

In some embodiments, the method further comprises administering an effective amount or a subtherapeutic amount of an additional therapeutic agent contemporaneously with or after the administering of the targeted activated T cell. In some embodiments, the additional therapeutic agent is a chemotherapeutic agent or an anti-neoplastic agent. As used herein, the term “subtherapeutic” refers to an amount of a pharmaceutical drug or agent that is insufficient to achieve the desired and/or anticipated therapeutic result/outcome upon administration to an average and/or typical subject (e.g., average size, taking no contraindicated pharmaceutical agents, having a similar reaction to the dose as a majority of the population, etc.). U.S. Food and Drug Administration (FDA) recommended dosages are indicative of a therapeutic dose administered alone for the treatment of a disease (e.g., cancer). In some embodiments, the subtherapeutic amount of the additional therapeutic agent is a lower amount as compared to an amount of the additional therapeutic agent administered to a subject when the additional therapeutic agent is administered to a subject by itself.

In some embodiments, the presently disclosed subject matter provides a method for treating a cancer in a subject in need thereof, the method comprising administering to the subject an effective amount of a composition comprising a targeted activated T cell, such as a bispecific antibody (BiAb) armed activated T cell (BAT), which selectively binds a cell of the cancer in the subject; and administering an effective amount or a subtherapeutic amount of an additional therapeutic agent to the subject, to thereby treat the cancer in the subject. In some embodiments, the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid or liquid tumor.

In some embodiments, the effective amount or the subtherapeutic amount of the additional therapeutic agent is administered contemporaneously with or after the administering of the targeted activated T cell. In some embodiments, the additional therapeutic agent is a chemotherapeutic agent or an anti-neoplastic agent. In some embodiments, the subtherapeutic amount of the additional therapeutic agent is a lower amount as compared to an amount of the additional therapeutic agent administered to a subject when the additional therapeutic agent is administered to a subject by itself.

In some embodiments, the presently disclosed subject matter provides a pharmaceutical composition comprising, consisting essentially of, or consisting of an effective amount of a targeted activated T cell, such as a bispecific antibody armed activated T cell (BAT), wherein the targeted activated T cell selectively binds a cell of the cancer in the subject. In some embodiments, the pharmaceutical composition is for use in a method for treating a cancer in a subject in need thereof, alone or in combination with an effective amount or a subtherapeutic amount of an additional therapeutic agent. In some embodiments, the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid or liquid tumor.

In some embodiments, the presently disclosed subject matter provides for the use of an effective amount of a targeted activated T cell, such as a bispecific antibody armed activated T cell (BAT), wherein the targeted activated T cell selectively binds a cell of the cancer in the subject, for the preparation of a medicament to treat cancer in a subject in need thereof. In some embodiments, the pharmaceutical composition is for use in a method for treating a cancer in a subject in need thereof, alone or in combination with an effective amount or a subtherapeutic amount of an additional therapeutic agent. In some embodiments, the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid or liquid tumor.

In some embodiments, the subject is a mammalian subject. In some embodiments, the composition comprising the targeted activated T cell and/or the additional therapeutic agent is/are adapted for administration for the treatment of a subject by intravenous administration, intrathecal injection, peritoneal injection, or direct injection into the tumor or surrounding tumor site.

In some embodiments, the cancer is a drug resistant cancer or drug sensitive cancer. In some embodiments, the cancer is a cancer characterized by the presence of or as a solid tumor or liquid tumor, or is a cancer of hematologic origin. Thus, in some embodiments, the cancer is selected from the group comprising, but not limited to, pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma. In some embodiments, the drug resistant cancer is selected from the group comprising, but not limited to, pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma. In some embodiments, the drug sensitive cancer is selected from the group comprising, but not limited to, pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma.

In some embodiments, the terms “chemotherapeutic agent” and “anti-neoplastic agent” refer to drugs (i.e., chemical compounds or small molecules) or prodrugs known to, or suspected of being able to treat a cancer (i.e., to kill cancer cells, prohibit proliferation of cancer cells, or treat a symptom related to cancer). In some embodiments, the terms “chemotherapeutic” or “anti-neoplastic agent”, as used herein refers to a natural or non-natural (i.e., synthetic) small molecule that is used to treat cancer and/or that has cytotoxic ability. Such more traditional or conventional chemotherapeutic agents or anti-neoplastic agents can be described by mechanism of action or by chemical compound class, and can include, but are not limited to, alkylating agents (e.g., melphalan, temozolomide), anthracyclines (e.g., doxorubicin), cytoskeletal disruptors (e.g., paclitaxel), epothilones, histone deacetylase inhibitors (e.g., vorinostat), inhibitors of topoisomerase I or II (e.g., irinotecan or etoposide), kinase inhibitors (e.g., bortezomib, imatinib, etc.), nucleotide analogs or precursors thereof (e.g., methotrexate), peptide antibiotics (e.g., bleomycin), platinum based agents (e.g., cisplatin or oxaliplatin), retinoids (e.g., tretinoin), and vinka alkaloids (e.g., vinblastine). In some embodiments, the chemotherapeutic agent is an antibody (e.g., a human, chimeric, or humanized antibody) or fragment thereof.

In some embodiments, the additional therapeutic agent is an additional cancer treatment. In some embodiments, the additional cancer treatment is selected from the group comprising surgery, radiotherapy, toxin therapy, immunotherapy, cryotherapy and gene therapy. In some embodiments, the immunotherapy agent is selected from the group comprising an anti-CD52 antibody, an anti-CD20 antibody, an anti-CD20 antibody, anti-CD47 antibody an anti-GD2 antibody, a radiolabeled antibody, an antibody-drug conjugate, a cytokine, polysaccharide K and a neoantigen; optionally wherein said cytokine is an interferon, an interleukin, or tumor necrosis factor alpha (TNF-α), further optionally where said cytokine is selected from the group comprising IFN-α, INF-γ, IL-2, IL-12 and TNF-α. In some embodiments, the immunotherapy agent is selected from the group comprising Alemtuzumab, Ofatumumab, Rituximab, Zevalin, Adcetris, Kadcyla and Ontak. In some embodiments, the immunotherapy agent is selected from the group comprising a PD-1 inhibitor, a PD-L1 inhibitor, a CTLA-4 inhibitor, an IDO inhibitor, and a CCR7 inhibitor.

Any suitable or desired approach for producing activated T cells as would be apparent to one of ordinary skill in the art upon a review of the instant disclosure can be employed. Representative approaches are provided in the EXAMPLES. In some embodiments, the targeted activated T cells are produced from an apheresis product. In some embodiments, the targeted activated T cells are produced from an apheresis product by anti-CD3 stimulation (such as through the use of a soluble OKT3 dose of 20 ng/ml) in the presence of IL-2, optionally at a range of about 20 to about 200 IU/ml, In some embodiments, co-stimulated T cells are produced from an apheresis product by co-stimulation with anti-CD3/anti-CD28 coated beads in the presence or absence of IL-2 (5-200 IU/ml, optionally 20-200 IU/ml, including 5, 10, 15, 20, 25, 30, 35, 40, 45, 50, 55, 60, 65, 70, 75, 80, 85, 90, 95, 100, 105, 110, 115, 125, 130, 135, 140, 145, 150, 155, 160, 165, 170, 175, 180, 185, 190, 195, 200 IU/ml), such as at bead to cell ratios from 1:3 to 3:1. Alternatively, co-stimulated T cells or T cell subsets are co-stimulated with anti-CD3/anti-CD2/anti-CD28 coated beads in the presence or absence of IL-2, with IL-2 in the amounts mentioned immediately above when present. Representative techniques are also disclosed in U.S. Pat. No. 7,763,243, U.S. Patent Application Publication No. 2018/0282693, U.S. Patent Application Publication No. 2018/0243341, and U.S. Patent Application Publication No. 2019/0343954, each of which is hereby incorporated by reference in its entirety.

As used herein, the term “targeted activated T cell” refers to a T cell (and in some cases other lymphocytes) that has/have been made selective for a target antigen, such as by being stimulated with antigenic material, such as an antigen from a cancer. The targeted activated T cell can be autologous or allogeneic to the subject. In accordance with the presently disclosed subject matter, any antigen expressed on the cell surface of a solid tumor or cancer of hematologic origin can be used to target a T cell or other cell, such as with bispecific antibody. Representative antigens include but are not limited to, EGFR, HER2, GD2, CD19, CD20, CD22, CD123, SLAMF7, CD38, SASB1, wntl, PMEL17 and CEA. Further, targeted activated T cells can be made selected for a target antigen by a T cell effector. An example of a T cell effector in accordance with the presently disclosed subject matter is a bispecific antibody. However, any suitable or desired T cell or T cell effector as would be apparent to one or ordinary skill in the art upon a review of the instant disclosure can be employed. In some embodiments, the T cell or T cell effector is selected from the group comprising peripheral blood mononuclear cells, unfractionated CD3+T cells, CD4+ T cells, CD8+ T cells, and combinations thereof. By way of example and not limitation, other T cell or T cell effectors include but are not limited to CD45RO+ T cells, CD45RA+ T cells, and CD3+CD56+ T cells, Chimeric Antigen Receptor T-cells (CAR-T), NK cells such as but not limited to Tumor infiltrating lymphocytes (TILs), cytotoxic T cells induced by viral or other antigen stimulation (CMV-specific cytotoxic T cells), bionic T cells engaging a specific cancer of hematologic origin (e.g., a leukemia) or solid tumors. Thus, in accordance with the presently disclosed subject matter a “targeted activated T cell” includes any other type of targeted T cells or targeted NK cells. Particular examples include CAR-T cells that specifically target solid tumor or liquid tumor antigens (e.g., from a cancer of hematologic origin), armed CAR-T cells (any CAR-T directed at for example CD19 armed with anti-CD3 x anti-Her2 BiAb that kills breast cancer) and NK cells of any type that are armed with BiAb to kill solid tumor targets. Stated another way, in some embodiments, any effector that can be targeted by CAR can be redirected by BiAb to sensitize a cancer to chemotherapy.

CAR-T involve engineered receptors which graft an arbitrary specificity onto a T cell. Typically, these receptors are used to graft the specificity of a monoclonal antibody (e.g., CD19) onto the T cell, with transfer of their coding sequence facilitated by retroviral vectors. These artificial T cell receptors allow grafting of nearly any specificity to T cells. This allows generation of large numbers of specific T cells without laborious selection and expansion procedures (see, for example, Pule, Finney and Lawson, Cytotherapy, 5 (3): 211-26, (2003) and Sadelain et. al., Nature, 545(7655):423-431 (2017). CAR-T are constructed by linking an antigen binding domain, usually a single chain variable fragment (scFv), to an intracellular T cell signaling domain such as CD3-(first generation CAR-T), and currently also including one or two co-stimulatory domains (second/third-generation CAR-T). The specific binding of CAR-T cells occurs in a non-MHC restriction manner, yet antigen binding results in T cell activation. Tumor infiltrating lymphocytes (TILs) are a type of white blood cells found in tumors. TILs are CD3+ cells that display activated natural killer cell (ANK) activity, but are more effective killers than ANK on a per cell basis (Rosenberg et al., Science 233:1318-1321 (1986)). Therapeutic TILs are a preparation of cells, comprising autologous tumor infiltrating lymphocytes that are manipulated in vitro and upon administration in vivo, re-infiltrate the tumor to initiate tumor cell lysis. To prepare therapeutic TILs in vitro, therapeutic tumor-infiltrating lymphocytes (TILs) can be isolated from tumor tissue and cultured with lymphokines such as IL-2; the TILs are then reinfused into the patient, where, after re-infiltration of the tumor, they may induce lysis of tumor cells and tumor regression. Bionic T cells are headless CAR T cells that has all the transmembrane and intracellular domains of the CAR without single chain variables fragment (scFv) of the specific antibody to target specific antigen. Since bionics do not have the scFv, they can be armed with bispecific antibodies to target any antigen on cancer cells without the need of re-engineering the CAR T cells to target specific antigen on cancer cells.

In accordance with aspects of the presently disclosed subject matter, in vivo use of activated T cells can vaccinate endogenous T cells of a subject to make immune responses as disclosed herein, such as in the EXAMPLES. Thus, in accordance with aspects of the presently disclosed subject matter BAT sensitization, or other activated T cell sensitization followed by chemotherapy or other therapeutic agent not only enhances the killing of cancer cells, but also provides more cancer antigens to immunize the endogenous immune system. Stated another way, the increase of killing with the immunosensitization in accordance with the presently disclosed subject matter leads to “vaccinating” the subject against their own cancer.

Targeted activated T cells are prepared and maintained in any suitable medium as would be apparent to one of ordinary skill in the part upon a review of the instant disclosure. In some embodiments, the culture comprises a basal medium. Other representative media and media ingredients are described in the EXAMPLES and/or would be apparent to one of ordinary skill in the art upon a review of the instant disclosure, including but not limited to known and/or commercially available media. By way of example and not limitation, other media and media components include but are not limited to RPMI 1640, Ex vivo 15, Ex Vivo 20, Aim V, CTS OpTmizer T-Cell Expansion SFM, LymphoONE, and/or other T cell culture or equivalent and other complete media in the presence or absence of serum, such as about 2 to about 10% fetal calf serum or human serum, including about 2%, about 3%, about 4%, about 5%, about 6%, about 7%, about 8%, about 9%, and about 10% serum, or artificial serum components (serum free media). In some embodiments, the culture comprises a media comprising RPMI1640 supplemented with 2% human serum. Commercial sources for media include Thermo Fisher Scientific (Hampton, N.H.), MilliporeSigma (Burlington, Mass.) and Sigma-Aldrich (St. Louis, Miss.). Ex vivo 15 and Ex vivo 20 are commercially available under the trademarks X-VIVO™ 15 and X-VIVO™ 20 (Lonza Walkersville, Inc., Walkersville, Md.); Aim V is commercially available under the trademark AIM V™ (Life Technologies Corporation, Carlsbad, Calif.), CTS OpTmizer T-Cell Expansion SFM is commercially available under the trademark CTS™ OpTmizer™ T Cell Expansion SFM (Life Technologies Corporation, Carlsbad, Calif.), and LymphoOne is commercially available under the trademark LYMPHOONE™ (Takara Bio Inc., Kusatsu, Japan).

Any suitable or desired bispecific antibody as would be apparent to one of ordinary skill in the art upon a review of the instant disclosure can be employed. In some embodiments, the bispecific antibody used to arm the targeted activated T cell is selected from the group including but not limited to a chemically heteroconjugated bispecific antibody or recombinant bispecific antibodies of any configuration (e.g., univalent, bivalent, or multi-valent bispecific antibodies directed at T cells and at the cancer or tumor antigen). In some embodiments, the bispecific antibody can be directed to any antigen expressed on the cell surface of a solid tumor or cancer of hematologic origin. Commonly expressed antigens include but are not limited to, EGFR, HER2, GD2, CD19, CD20, CD22, CD123, SLAMF7, CD38, SASB1, wntl, PMEL17 and CEA. In some embodiments, the BAT comprises anti-CD3 x anti-EGFR BiAb, anti-CD3 x anti-HER2 BiAb, anti-CD3 x anti-GD2 BiAb, anti-CD3 x anti-CD20 BiAb, anti-CD3 x anti-SLAMF7 BiAb, or other targets. See, for example, U.S. Pat. No. 7,763,243, U.S. Patent Application Publication No. 2018/0282693, U.S. Patent Application Publication No. 2018/0243341, and U.S. Patent Application Publication No. 2019/0343954, each of which is hereby incorporated by reference in its entirety. Additional examples are also described, such as in Thakur et al., Oncoimmunology. 2018 Aug. 27; 7(12); Vaishampayan et al., Prostate Cancer. 2015; 2015:285193, Epub 2015 Feb. 23; Lum et al., Clin Cancer Res. 2015 May 15; 21(10):2305-14, Epub 2015 Feb. 16; Lum et al., Biol Blood Marrow Transplant. 2013 June; 19(6):925-33, Epub 2013 Mar. 22; Zitron et al., BMC Cancer. 2013 Feb. 22; 13:83; Yankelevich et al., Pediatr Blood Cancer. 2012 Dec. 15; 59(7):1198-205. In some embodiments, the BiAb used to arm the targeted activated T cell is a chemically heteroconjugated bispecific antibody or a recombinant bispecific antibody of any configuration.

The presently disclosed subject matter is also directed to methods of administering the compositions of the presently disclosed subject matter to a subject.

Pharmaceutical compositions in accordance with the presently disclosed subject matter are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal approaches.

In accordance with one embodiment, a method for treating a subject in need of such treatment is provided. The method comprises administering a pharmaceutical composition comprising at least one composition of the presently disclosed subject matter to a subject in need thereof. Compositions provided by the methods of the presently disclosed subject matter can be administered with known compounds or other medications as well.

The presently disclosed subject matter encompasses the preparation and use of pharmaceutical compositions for treatment of the diseases and disorders disclosed herein. Such a pharmaceutical composition can consist of the active ingredient alone, in a form suitable for administration to a subject, or the pharmaceutical composition can comprise the active ingredient and one or more pharmaceutically acceptable carriers, one or more additional ingredients, or some combination of these. The active ingredient can be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter can comprise at least one active ingredient, one or more acceptable carriers, and optionally other active ingredients or therapeutic agents.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions can also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) can be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter or pharmaceutical compositions comprising these compositions can be administered so that the compositions may have a physiological effect. Administration can occur enterally or parenterally; for example, orally, rectally, intracisternally, intravaginally, intraperitoneally, locally (e.g., with powders, ointments or drops), or as a buccal or nasal spray or aerosol. Parenteral administration is an approach. Particular parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, and direct application to the target area, e.g., intratumoral injection, for example by a catheter or other placement device.

Where the administration of the composition is by injection or direct application, the injection or direct application can be in a single dose or in multiple doses. Where the administration of the compound is by infusion, the infusion can be a single sustained dose over a prolonged period of time or multiple infusions.

The formulations of the pharmaceutical compositions described herein can be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially and/or socially relevant mammals such as cattle, pigs, horses, sheep, cats, and dogs, birds including commercially and/or socially relevant birds such as chickens, ducks, geese, parrots, and turkeys.

A pharmaceutical composition of the presently disclosed subject matter can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition can comprise between 0.1% and 100% (w/w) active ingredient. It can generally be stated that a pharmaceutical composition comprising the T cells described herein may be administered at a dosage of 10⁴ to 10⁹ cells/kg body weight, optionally 10⁵ to 10⁶ cells/kg body weight, including all integer values within those ranges. T cell compositions may also be administered multiple times at these dosages. The cells can be administered, for example, by using infusion techniques that are commonly known in immunotherapy (see, e.g., Rosenberg et al., New Eng. J. of Med. 319:1676, 1988). The optimal dosage and treatment regime for a particular subject can readily be determined by one skilled in the art of medicine by monitoring the subject for signs of disease and adjusting the treatment accordingly.

Controlled- or sustained-release formulations of a pharmaceutical composition of the presently disclosed subject matter can be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents;

stabilizing agents; and pharmaceutically acceptable polymeric or hydrophobic materials. Other “additional ingredients” which may be included in the pharmaceutical compositions of the presently disclosed subject matter are known in the art and described, for example in Gennaro (1990) Remington's Pharmaceutical Sciences, 18th ed., Mack Pub. Co., Easton, Pa., United States of America and/or Gennaro (ed.) (2003) Remington: The Science and Practice of Pharmacy, 20th edition Lippincott, Williams & Wilkins, Philadelphia, Pa., United States of America, each of which is incorporated herein by reference.

The compositions may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The presently disclosed subject matter also includes a kit comprising the compositions of the presently disclosed subject matter and an instructional material which describes administering the composition to a cell or a tissue of a subject. In some embodiments, this kit comprises a (in some embodiments sterile) solvent suitable for dissolving or suspending the composition of the presently disclosed subject matter prior to administering the compound to the subject and/or a device suitable for administering the composition such as a syringe, injector, or the like or other device as would be apparent to one of ordinary skill in the art upon a review of the instant disclosure.

As used herein, an “instructional material” includes a publication, a recording, a diagram, or any other medium of expression which can be used to communicate the usefulness of the composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter can, for example, be affixed to a container which contains a composition of the presently disclosed subject matter or be shipped together with a container which contains the composition. Alternatively, the instructional material can be shipped separately from the container with the intention that the instructional material and the composition be used cooperatively by the recipient.

In accordance with the presently disclosed subject matter, as described above or as discussed in the EXAMPLES below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al. (1989) Molecular Cloning: A Laboratory Manual, Cold Spring Harbor Laboratory Publications, Cold Spring Harbor, N.Y., United States of America; Glover (1985) DNA Cloning: A Practical Approach. Oxford Press, Oxford; Gait (1984) Oligonucleotide Synthesis: A Practical Approach, IRL Press, Oxford, England; Harlow & Lane, 1988, Antibodies, A Laboratory Manual, Cold Spring Harbor Publications, New York; Roe et al. (1996) DNA Isolation and Sequencing: Essential Techniques, John Wiley, New York, N.Y., United States of America; and Ausubel et al. (1995) Current Protocols in Molecular Biology, Greene Publishing.

### III.C. Antibody Formats and Preparation Thereof

Any suitable bispecific antibody and technique for the production thereof as would be apparent to one of ordinary skill in the art upon a review of the instant disclosure falls within the scope of the presently disclosed subject matter. In some embodiments, the presently disclosed subject matter employs bispecific antibodies (BiAbs) produced by chemical joining of two monoclonal antibodies. Examples of bispecific antibodies and techniques for producing bispecific antibodies are known the art and have been described in several reviews, along with their respective cancer antigens and T cell antigens. Representative reviews include Thakur, A., and Lum, L. G.: Cancer therapy with bispecific antibodies: Clinical experience. Current Opinion and Molecular Therapeutics 12:340-349, 2010; Lum, L. G., and Thakur, A.: Bispecific Antibodies for Arming Activated T Cells and Other Effector Cells for Tumor Therapy. Book Chapter in: Bispecific Antibodies. Kontermann, R. E. (ed). Germany: Springer Heidelberg, 2011, pp. 243-271; Lum, L. G., and Thakur, A.: Targeting T Cells with Bispecific Antibodies for Cancer Therapy: A Review. BioDrugs 25:365-379, 2011; and Thakur, A., Huang, M., Lum, L. G.: Bispecific antibody based therapeutics: Strengths and challenges. Blood Reviews, 2018 (Impact 6.6). Representative BiAbs include but are not limited to whole IgG-based BiAbs, trifunctional BiAbs, BiAb Format based on single-chain variable fragment. Representative U.S. patents relating to BiAbs and production thereof include U.S. Pat. Nos. 10,550,193; 10,519,247; 10,294,300; 10,239,951; and 10,179,819, each of which is herein incorporated by reference in its entirety.

In some embodiments, one or more antibodies or fragments thereof are used. In some embodiments, one or both antibodies are single chain, monoclonal, bi-specific, synthetic, polyclonal, chimeric, human, or humanized, or active fragments or homologs thereof. In some embodiments, the antibody binding fragment is scFV, F(ab′)₂, F(ab)₂, Fab′, or Fab.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments. In some embodiments, the specific binding molecule is a single-chain variable (scFv). The specific binding molecule or scFv may be linked to other specific binding molecules (for example other scFvs, Fab antibody fragments, chimeric IgG antibodies (e.g., with human frameworks)) or linked to other scFvs of the presently disclosed subject matter so as to form a multimer which is a multi-specific binding protein, for example a dimer, a trimer, or a tetramer. Bi-specific scFvs are sometimes referred to as diabodies. Fragments within the scope of the term “antibody” include those produced by digestion with various proteases, those produced by chemical cleavage and/or chemical dissociation and those produced recombinantly, so long as the fragment remains capable of specific binding to a target molecule (i.e., comprise at least one paratope).

Other representative patent documents disclosing techniques relating to antibody production include the following, all of which are herein incorporated by reference in their entireties: PCT International Patent Application Publication Nos. WO 1992/02190 and WO 1993/16185; U.S. Patent Application Publication Nos. 2004/0253645, 2003/0153043, 2006/0073137, 2002/0034765, and 2003/0022244; and U.S. Pat. Nos. 4,816,567; 4,946,778; 4,975,369; 5,001,065; 5,075,431; 5,081,235; 5,169,939; 5,202,238; 5,204,244; 5,225,539; 5,231,026; 5,292,867; 5,354,847; 5,436,157; 5,472,693; 5,482,856; 5,491,088; 5,500,362; 5,502,167; 5,530,101; 5,571,894; 5,585,089; 5,587,458; 5,641,870; 5,643,759; 5,693,761; 5,693,762; 5,712,120; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,548,640; 6,632,927; 6,639,055; 6,750,325; and 6,797,492.

### IV. Examples

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Introduction to Examples

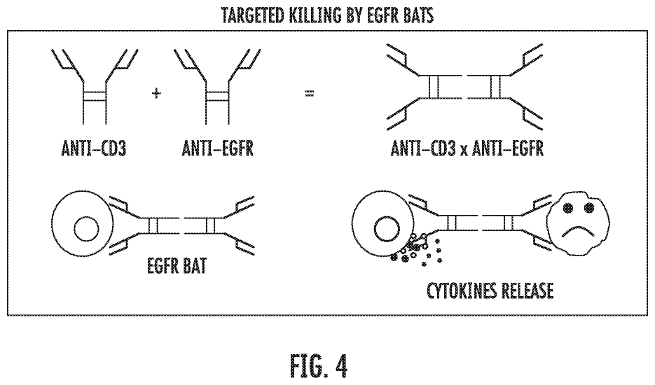

Chemotherapeutic (chemoT) responses of pancreatic cancer (PC) are short, and PC rapidly develops multidrug resistance with median overall survival of locally advanced PC and metastatic PC (MPC) patients of 7 months. The following EXAMPLES ask whether pre-targeting tumor cells with EGFR BATs can “sensitize” parental and drug-resistant tumor cell lines to subsequent chemotherapy while also reducing the dose of drug required to achieve effective cytotoxicity. EGFR BATs are produced by activating T cells with OKT3, expanding them in IL-2, and arming the activated T cells (ATCs) with EGFR bispecific antibody (EGFRBi). The EGFRBi is generated via chemical conjugation of anti-CD3 to anti-EGFR (Zitron et al., BMC Cancer. 2013 Feb. 22; 13:83). Arming of T cells with EGFRBi turns each T cell into a non-MHC restricted EGFR-specific cytotoxic T lymphocyte. Upon engagement of a tumor cell, an array of cytokines is released, which leads to the destruction of the tumor target and stimulation of the endogenous immune system. See FIG. 4.

Thus, an aspect of these EXAMPLES was to determine whether pancreatic cancer cells can be targeted by anti-CD3 x anti-EGFR BATs (EGFR BATs) or anti-CD3 x anti-HER2 BATs (HER2 BATs; Thakur et al., Oncoimmunology. 2018 Aug. 27; 7(12):e1500672, eCollection 2018; Vaishampayan et al., Prostate Cancer. 2015; 2015:285193, Epub 2015 Feb. 23; Lum et al., Clin Cancer Res. 2015 May 15; 21(10):2305-14, Epub 2015 Feb 16) and whether EGFR BATs can sensitize pancreatic cancer cells for enhanced chemotherapeutic responses. Cisplatin (CIS) and/or gemcitabine (GEM) resistant L3.6, PANC-1, and MiaPaCa-2 cell lines were generated. Parental and resistant cell lines were targeted by ATC or BATs. Parental and resistant cell lines were either primed with ATC or BATs followed by retargeting with the same bispecific antibody armed ATC. Parental and resistant cell lines were primed with ATC or EGFR BATs followed by a dose titration of cisplatin.

### Material and Methods

Cell culture and maintenance. Human pancreatic carcinoma cell lines MiaPaCa-2 and PANC-1 were obtained from America Type Culture Collection (ATCC) and maintained in Dulbecco's Modified Eagle Media (DMEM) (Sigma-Aldrich) supplemented with 10% fetal bovine serum (Atlanta Biologicals), 60 μg/mL penicillin and 100 μg/mL streptomycin (Lonza), and 6 mM L-glutamine (Hyclone). Activated T cells and BATs were maintained in Roswell Park Memorial Institute media (RPMI-1640) supplemented with 10% fetal bovine serum (Atlanta Biologicals), 60 μg/mL penicillin and 100 μg/mL streptomycin (Lonza), and 6 mM L-glutamine (Hyclone). Cell were grown in a humidified incubator at 37° C. at 5% CO₂.

Determining IC₅₀ concentrations of cisplatin in MiaPaCa-2 and PANC-1. The 50% inhibitory concentration (IC₅₀) for cisplatin was evaluated using the MTT (3-(4,5-dimethylthiazol-2-yl)-2,5-diphenyltetrazolium bromide) colorimetric assay (Promega). MiaPaCa-2 and PANC-1 cells were trypsinized, washed, and incubated overnight in a humidified incubator. Each condition was performed in quadruplicate with 3×10³ cells/well on a 96-well flat bottom plate. Cells were subsequently treated for 72 hours in the absence or presence of cisplatin ranging from 0.2 μM to 75 μM. After drug treatment, 15 μL of the MTT dye solution was added to each well and incubated for 4 hours in a humidified incubator. The reaction was stopped by adding 100 μL of solubilization solution and the plate was incubated for an additional hour at room temperature. The wells were mixed and the absorbance was measured at 570 nm using a Synergy HTX Plate Reader. The % inhibition was calculated using the following equation:

$$\% Inhibition = 100 - ( Treated Absorbance \times 100 )$$

$$Untreated Absorbance$$

The % inhibition values were plotted against their respective cisplatin concentrations and the IC₅₀ values for both cell lines were extrapolated accordingly.

Determination of Cytotoxicity by ⁵¹Cr Release Assay. Cytotoxic activity of BATs against specific targets was assess by ⁵¹Cr release assay in 96-well flat-bottom microtiter plates. Briefly, ATC, EGFR BATs or HER2 BATs were plated in triplicate onto 4 x 10⁴ cells/well target cells (MiaPaCa-2, L3.6 or PANC-1 and their CIS or GEM resistant derivatives) at different effector:target (E/T) ratios and ⁵¹Cr release was measured after 18-20 hours. Percent specific cytotoxicity was calculated using the following formula:

(experimental cpm−spontaneous cpm)/(maximum cpm−spontaneous cpm)×100.

Generating cisplatin resistant L3.6, MiaPaCa-2 and PANC-1 cell lines. Cisplatin (CIS) and Gemcitabine (GEM) resistant L3.6, MiaPaCa-2 and PANC-1 were generated by culturing the tumor cells in the presence of their respective IC₅₀ concentrations of CIS (L3.6, 16.0 μM; MiaPaCa-2, 18.6 μM; PANC-1, 14.0 μM) or Gem (L3.6, 12.0 μM; MiaPaCa-2, 15.6 μM) for 72 hours. After treatment, the remaining cells were washed and incubated in culture media until near confluency was reached. The cell lines were then subcultured and exposed to one tenth of their respective IC₅₀ concentrations. The cell lines were continually grown and subcultured in this manner until they were stable in one tenth of their IC₅₀ concentration. Once stable, the cell lines were subcultured and exposed to double the previous concentration. This process was repeated continuously until the cells were being grown in half of their respective IC₅₀ concentration.

BATs Immunosensitization Assays using the xCelligence RTCA. The response of BATs immunosensitized native and resistant MiaPaCa-2 and PANC-1 cell lines to cisplatin was evaluated using the cell-impedance based xCELLigence RTCA MP system (ACEA Biosciences) and the respective 96-well E-Plates (ACEA Biosciences). The xCELLigence RTCA MP system was stored inside a humidified incubator and all manipulations to the 96-well E-Plate were performed inside a laminar flow hood. Prior to each experiment, a media-only impedance check was performed by adding 50 μL of cell culture media to every well of the 96-well E-Plates and then measuring the background impedance as a unit of Cell Index. After the initial background check, the adherent cell lines were trypsinized, washed, and seeded at 1×10⁴ cells/well (MiaPaCa-2) and 5×10³ cells/well (PANC-1). Seeded plates were kept for 30 minutes at ambient temperature to allow for even distribution and settling of tumor cells. After thirty minutes, the plates were transferred to the xCELLigence RTCA MP system and incubated overnight. The RTCA MP system was programmed to collect Cell Index data points at 15 minute intervals. The next day, cryopreserved OKT3 activated T cells were thawed and armed with a chemically conjugated OKT3 x EGFR bispecific antibody at 50 ng/1×10⁶ cells for 15 minutes to generate EGFR BATs. These EGFR BATs were washed twice with culture media and supplemented with 100 IU/mL IL-2. Data acquisition was paused and the E-Plate was removed from the RTCA MP. The EGFR BATs were seeded onto the E-Plate at an E:T ratio of 2:1 for native tumor cells and 1:2 for cisplatin resistant tumor cells and transferred to the RTCA MP. Data acquisition was resumed and the E-Plate was incubated overnight. The next day, data acquisition was paused and the E-Plate was removed. The supernatant from the E-Plate was decanted and each well was washed once with culture media. Immediately following, DMEM supplemented with cisplatin at the respective IC₅₀ concentration, half of the IC₅₀ concentration, and a quarter of the IC₅₀ concentration for each cell line was added to the E-Plate. The E-Plate was transferred to the RTCA MP and the experiment was resumed. Data acquisition continued for approximately 72 hours. Data output was normalized to a common Delta Time, which converted Cell Index to Delta Cell Index (DCI) and established a common starting point for each condition. The % cytotoxicity was calculated for the time point immediately preceding Delta Cell Index saturation of the untreated tumor cells using the following equation:

$$\% Cytotoxicity = ( {DCI}_{TumorAlone} - {DCI}_{Treatment} ) \times 100$$

$${DCI}_{TumorAlone}$$

Where:

DCI_{Tumor Alone} is the average Delta Cell Index between replicates of untreated tumor cells; and

DCI_{Treatment} is the average Delta Cell index between replicates of BATs and/or cisplatin treated tumor.

### Example 1

### BATs can Target Chemoresistant Pancreatic Cancer Cell Lines

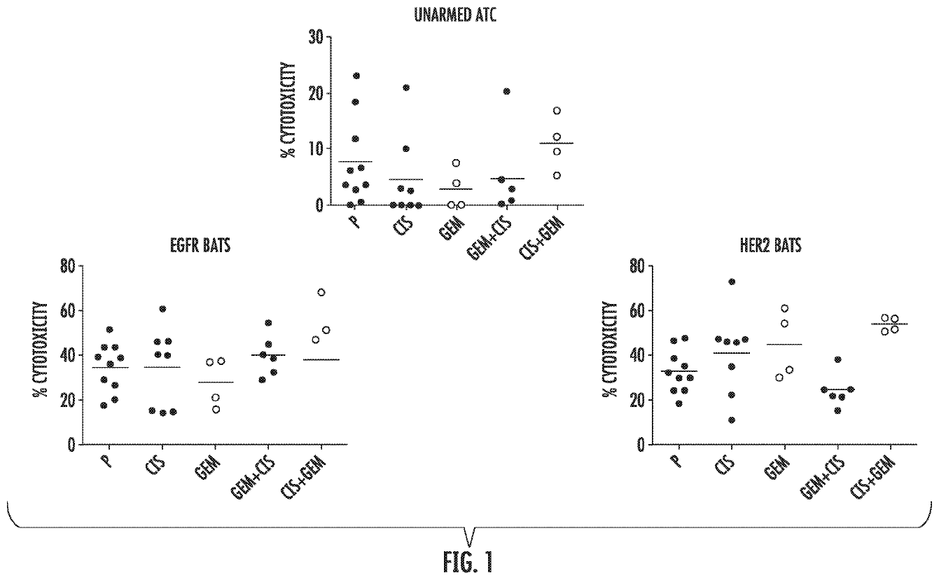

CIS or GEM resistant L3.6, PNAC-1 and MiaPaCa-2 cell lines and dual (CIS and Gem) resistant L3.6 cells were generated. For dual resistant cells, GEM resistant cells were used to generate dual GEM followed by CIS (GEM/CIS) resistant derivative. Similarly, CIS resistant cells were used to generate dual CIS followed by GEM resistant derivative were followed by gemcitabine (CIS/GEM) resistant drug, resistant L3.6 cell lines. It was determined whether EGFR BATs can target parental and resistant cell lines. ⁵¹Cr release assay was used to determine the cytotoxicity by EGFR BATs or HER2 BATs (n=6) against parental L3.6 pancreatic cancer cell line and its GEM resistant or CIS resistant derivatives. The presently disclosed data show that GEM, CIS, GEM/CIS or CIS/GEM resistant pancreatic cancer cells were effectively targeted by both EGFR BATs and HER2 BATs (FIG. 1).

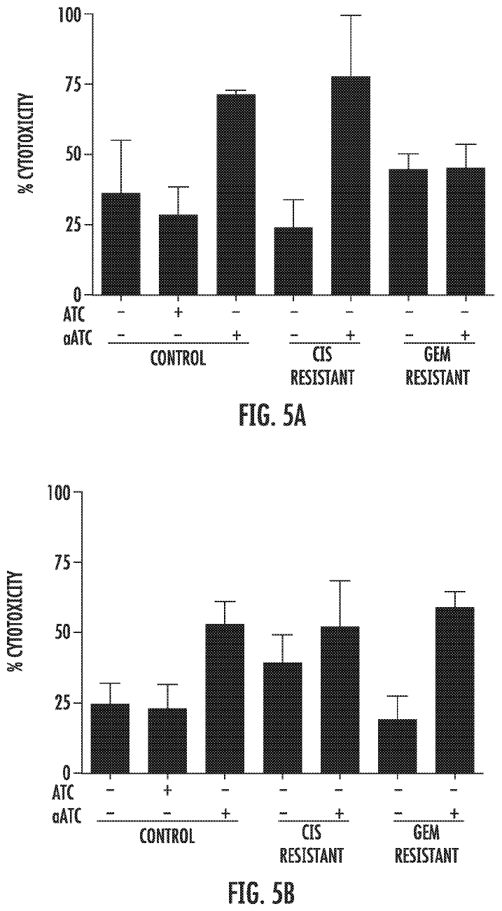

Referring now to FIGS. 5A and 5B, ⁵¹Cr release was used to test whether BATs at E:T 25:1 can be used to target then retarget parental and drug-resistant L3.6. FIG. 5A shows parental and drug-resistant L3.6 cells pretreated with ATC or bispecific antibody armed ATC (aATC) followed by retreatment with EGFR BATs, while FIG. 5B shows parental and drug resistant L3.6 cells pretreated with ATC or aATC followed by retreatment with HER2 BATs. These data show that BATs can target and kill parental and drug-resistant pancreatic tumor cell lines. Further experiments tested whether BATs treatment augments tumor response to subsequent chemotherapy.

### Example 2

### Enhanced Chemo responsiveness of Pancreatic Cancer Cell Line after Priming with BATs

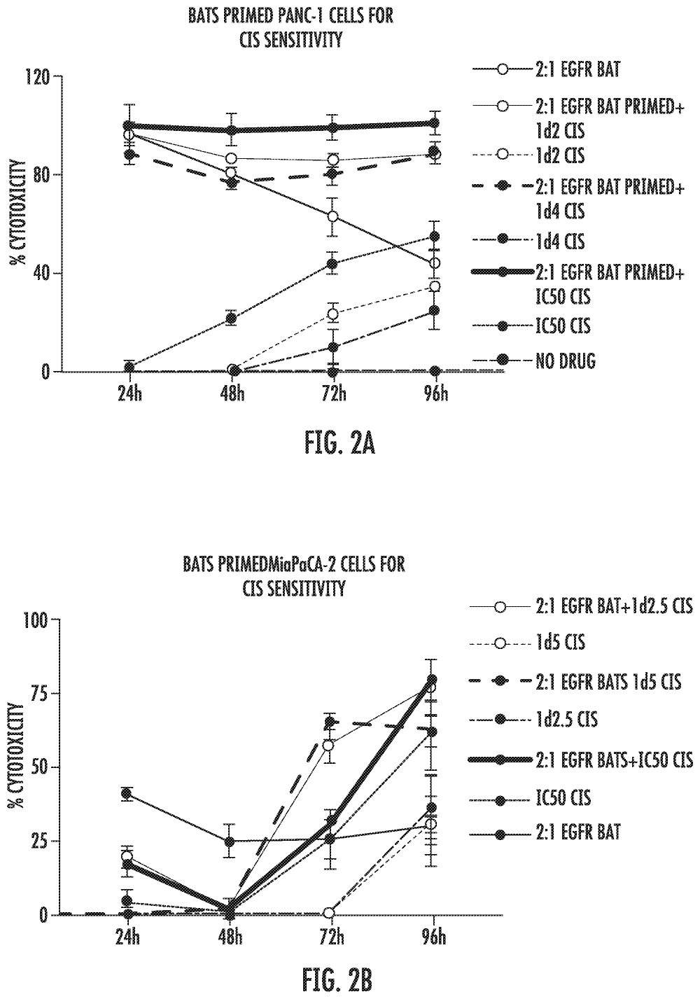

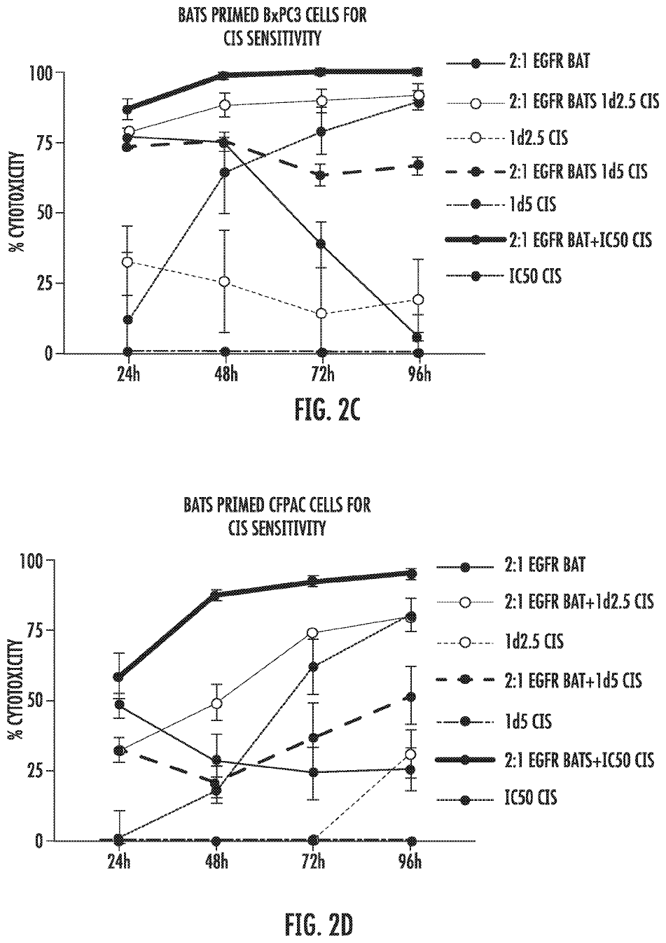

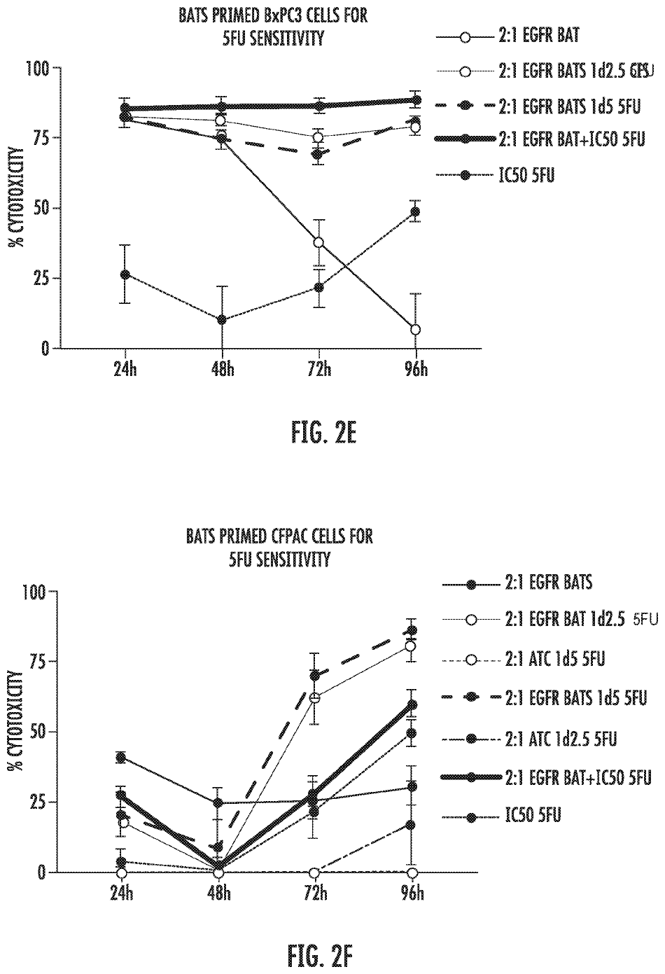

MiaPaCa-2, PANC-1 and BxPC-3 cells either primed with BATs followed by retargeting with 5 or 2.5 times lower concentrations of the IC₅₀ concentration of CIS (IC₅₀ concentration of MiaPaCa-2=18.6 PANC-1-14.0 CFPAC=10 μM and BxPC-3=6.9 μM) and 5FU (IC₅₀ concentration of CFPAC=5 μM and BxPC-3=14.6 μM) showed enhanced cytotoxicity compared to non-BATs primed cells. The mean cytotoxicity directed at MiaPaCa-2 increased with BATs priming at concentrations lower than the IC₅₀ (4.7 and 9.3 μM). At the 4.7 μM dose, cytotoxicity increased from CIS alone (31±15%) to BATs primed followed by CIS (57±17%), and BATs together with CIS (77±12%). In PANC-1 cells, there was low specific cytotoxicity with CIS concentrations at 3.5, 7.0, and 14 μM (10, 20 and 40%, respectively) at 72 hours. BATs priming before addition of CIS increased specific cytotoxicity to 80%, 82%, and 100% at CIS concentrations of 3.5, 7.0, and 14 μM, respectively, at 72 hours. In BxPC-3 cells, there was low specific cytotoxicity against BxPC-3 cells with CIS concentrations at 1.38, 2.76, and 6.9 μM that increased cytotoxicity after priming BxPC3 cells with BATs before addition of CIS from 0-30% to 70-80%. Likewise, there was no cytotoxicity in with CIS concentrations at 2.5, 5.0, and 10 μM but after priming of CFPAC-1 cells with BATs before addition of CIS increased specific cytotoxicity from 0% to 30-60%. Similar data was obtained for 5 fluorouracil (5FU) in BxPC-3 and CFPAC-1 cell lines. See FIGS. 2A-2F.

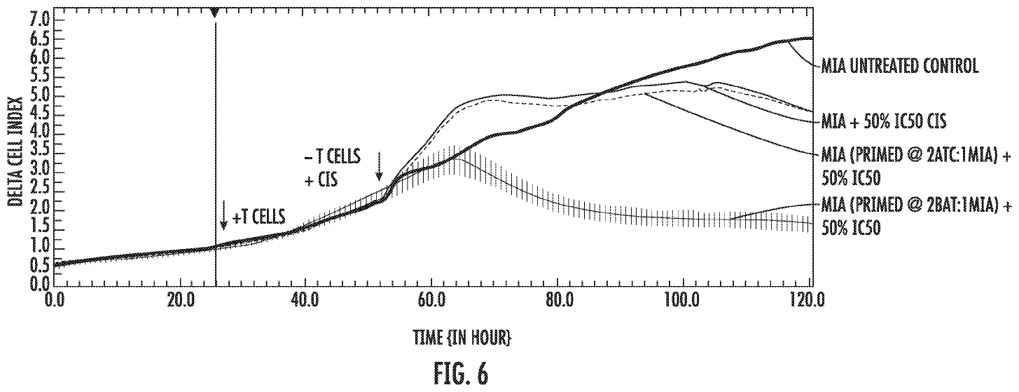

Referring now to FIG. 6, the xCelligence RTCA system was used to evaluate EGFR BATs sensitization of parental pancreatic cancer cell lines to chemotherapy. The vertical black line denotes the normalization point. The first arrow indicates when ATC or EGFR BATs were added, and the second arrow indicates when T cells were washed and CIS was introduced. Conditions were run in triplicate. Dashed lines depict standard deviation. IC₅₀ CIS: MiaPaCa-2=18.6 μM; PANC-1=14.0 μM. MiaPaCa-2 primed with EGFR BATs at E:T of 2:1 show enhanced sensitivity to CIS at half the IC₅₀ concentration over the unprimed control. With successful sensitization of parental MiaPaCa-2 at an E:T of 2:1 to CIS, further experiments tested the potency of lower E:T ratios.

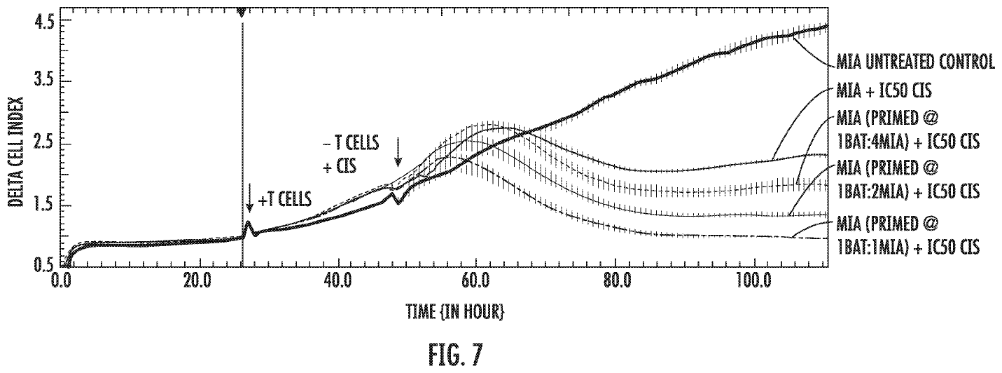

Referring to FIG. 7, parental MiaPaCa-2 were primed with EGFR BATs at various E:T ratios (1:1, 1:2, and 1:4) and then treated with CIS at the IC₅₀ concentration. The vertical black line denotes the normalization point. The first arrow indicates when EGFR BATs were added, and the second arrow indicates when EGFR BATs were washed and CIS was introduced. Dashed lines depict standard deviation. Conditions were run in triplicate. IC₅₀ CIS: MiaPaCa=18.6 μM. EGFR BATs priming at low E:T continually increased sensitivity to the cytotoxic effects of CIS at the IC₅₀ concentration. With data suggesting that EGFR BATs at low E:T continue to sensitize tumors at the IC₅₀ concentration, the next series of experiments evaluated how BATs priming augments CIS sensitivity in parental pancreatic cell lines by dose titrating CIS.

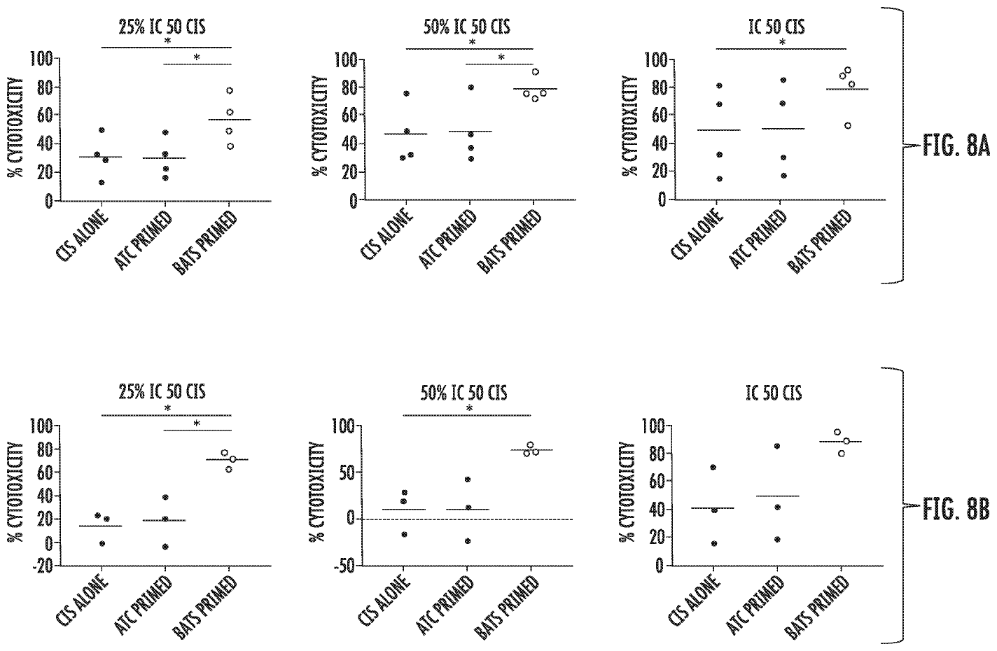

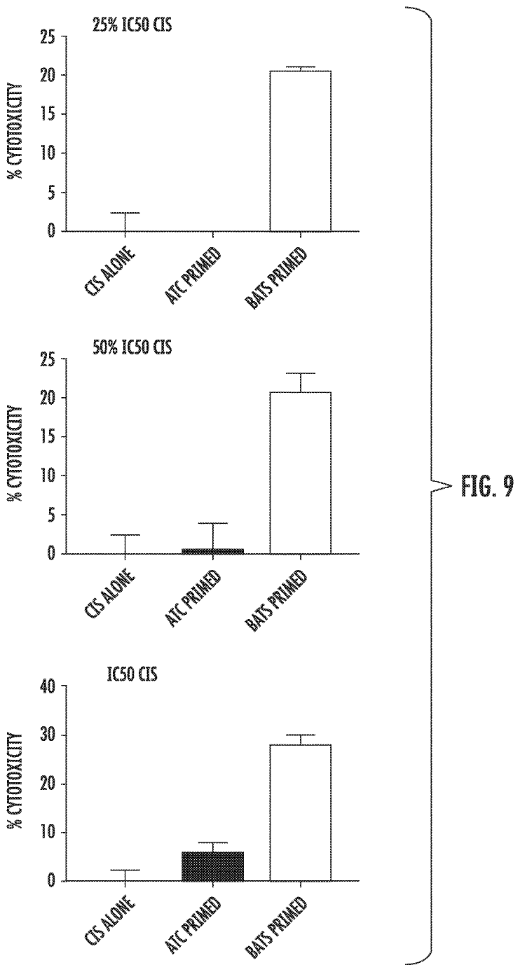

Referring to FIGS. 8A and 8B, the xCelligence system was used to test whether exposure of parental MiaPaCa-2 (FIG. 8A, n=4) or parental PANC-1 (FIG. 8B, n=3) to EGFR BATs at a 2:1 E:T increases CIS sensitivity when treated with 25%, 50%, or 100% the IC₅₀ concentration of CIS. Conditions were run in triplicate. Cell index (CI) readings from the xCelligence were taken 72 HR post chemotherapy normalized, and converted to % Cytotoxicity using the following formula: % Cytotoxicity=(CI tumor only−CI treated)/(CI tumor only)×100. IC₅₀ CIS: MiaPaCa-2 =18.6 μM; PANC-1 =14.0 μM. Paired t-test, *P<0.05. Cytotoxic effects of CIS were generally greater after priming when compared to CIS alone at and below IC₅₀ concentrations. With the ability of EGFR BATs to sensitize parental pancreatic cell lines to CIS, the next experiments tested the effect on CIS-resistant pancreatic cancer cells.

Referring to FIG. 9, using the xCelligence system, CIS-resistant MiaPaCa-2 were primed with EGFR BATs at an E:T of 1:2 before exposure to various concentrations of CIS.

The vertical black line denotes the normalization point. Cell index (CI) readings from the xCelligence system were taken 72 HR post chemotherapy, normalized, and converted to % Cytotoxicity using the following formula: % Cytotoxicity=(CI tumor only−CI treated)/(CI tumor only)×100. IC₅₀ CIS: MiaPaCa=18.6 μM. Conditions were run in triplicate. Cytotoxic effects of CIS appeared to be greater after priming when compared to CIS alone.

### Example 3

### Enhanced Chemo responsiveness of Chemoresistant PANC-1 Pancreatic Cancer Cell Line after Priming with BATs

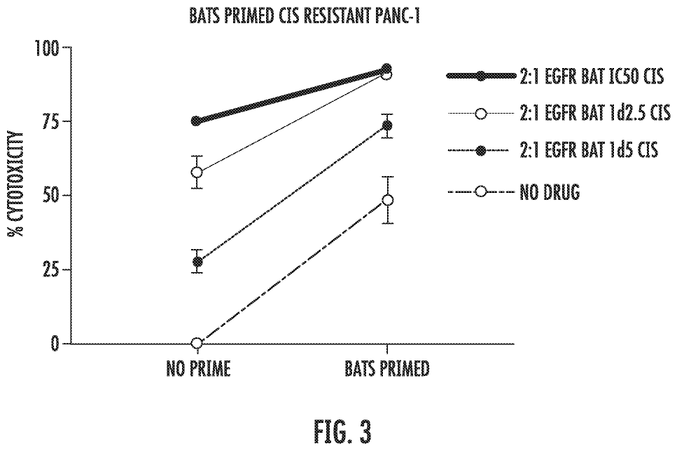

CIS PANC-1 cells the primed with BATs followed by retargeting with 5 or 2.5 times lower concentrations than the IC₅₀ concentration of CIS showed enhanced cytotoxicity compared to non-primed CIS resistant cells. The cytotoxicity was 25%, 56% and 74% at IC₅₀ concentration and at 5 and 2.5 times lower concentrations of IC₅₀ concentration of CIS at 72 hours. The mean cytotoxicity at CIS Resistant PANC-1 increased with BATs priming to 84%, 68%, 82% with IC₅₀ concentration and with 5 and 2.5 times lower concentrations of IC₅₀ dose of CIS at 72 hours (FIG. 3).

### Discussion of Examples 1-3

Pancreatic cancer has the worst survival rate of all cancers. Chemotherapeutic (ChemoT) responses of pancreatic cancer are short and rapidly develop multidrug resistance with dismal median overall survival (OS) in patients with locally advanced pancreatic and metastatic pancreatic cancer. This study asks whether pre-targeting tumor cells with EGFR BATs can “sensitize” drug resistant tumor cell lines so that subsequent treatment can not only kill the MDR PC lines but also decrease the doses of chemotherapy required to kill tumor cells. This BATs priming strategy augments clinical responses, decreases chemoT related toxicities, and improves overall survival for not only pancreatic cancer but for other solid tumors. While it is not desired to be bound by any particular mechanism of action, since Th1 cytokines are released during BATs mediated killing of chemoresistant tumor cells, it is likely that Th1 cytokines modulates the tumor microenvironment to enhance anti-tumor immune responses and sensitizes the tumor for enhanced chemotherapeutic responsiveness. In summary, it was shown that: 1) BATs can kill chemo resistant tumor cells; and 2) pretreatment or sensitization of the tumor with BATs lowers the threshold for effective cytotoxic doses of CIS against PANC-1. These data show that priming of drug sensitive or drug resistant cells with BATs can sensitize PANC-1 for enhanced responsiveness to chemotherapeutic drugs at much lower drug concentrations.

Thus, chemotherapeutic drug resistance of pancreatic cancer cells does not affect targeted killing by EGFR BATs or HER2 BATs. Priming of parental or drug-resistant cells with EGFR BATs followed by retargeting with CIS showed enhanced cytotoxicity at various doses of CIS. At a higher E:T of 2:1, the effective cytotoxic dose of CIS is reduced after EGFR BATs priming. EGFR BATs priming is effective at E:T ratios as low as 1:1, 1:2, and 1:4 for MiaPaCa-2 when treated with CIS at the IC₅₀ concentration.

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
1. Siegel, R. L., Miller, K. D. and Jemal, A. Cancer statistics, CA: A Cancer Journal for Clinicians, 68: 7-30, 2018.
2. Kang S P, Saif M W Pharmacogenomics and pancreatic cancer treatment. Optimizing current therapy and individualizing future therapy., J Pancreas 9: 251-266, 2008.
3. Manu Gnanamony, Christopher S. Gondi. Chemoresistance in pancreatic cancer: Emerging concepts. Oncol Lett. 13: 2507-2513, 2017.
4. Itamochi H, Kigawa J, Terakawa N. Mechanisms of chemoresistance and poor prognosis in ovarian clear cell carcinoma. Cancer Sci 99: 653-658, 2008.
5. Coley H M. Mechanisms and strategies to overcome chemotherapy resistance in metastatic breast cancer. Cancer Treat Rev 34: 378-390, 2017.
6. Aleksandra Adamska, Marco Falasca. ATP-binding cassette transporters in progression and clinical outcome of pancreatic cancer: What is the way forward? World J Gastroenterol. 24(29): 3222-3238, 2018.
7. Schinkel A H, Jonker J W. Mammalian drug efflux transporters of the ATP binding cassette (ABC) family: an overview. Adv Drug Deliv Rev. 55:3-29, 2003.
8. Kruh G D, Belinsky M G. The MRP family of drug efflux pumps. Oncogene. 22:7537-7552, 2003.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

What is claimed is:

1. A method for treating a cancer in a subject in need thereof, the method comprising administering to the subject an effective amount of a composition comprising a targeted activated T cell which selectively binds a cell of the cancer in the subject, to thereby treat the cancer in the subject.

2. The method of claim 1, wherein the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid tumor.

3. The method of claim 1 or claim 2, wherein the cancer is a drug resistant cancer or drug sensitive cancer.

4. The method of any one of claims 1-3, wherein the cancer is selected from the group consisting of pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma.

5. The method of any one of claims 1-4, further comprising administering an effective amount or a subtherapeutic amount of an additional therapeutic agent contemporaneously with or after the administering of the targeted activated T cell.

6. The method of claim 5, wherein the additional therapeutic agent is a chemotherapeutic agent or an anti-neoplastic agent.

7. The method of claim 5 or claim 6, wherein the subtherapeutic amount of the additional therapeutic agent is a lower amount as compared to an amount of the additional therapeutic agent administered to a subject when the additional therapeutic agent is administered to a subject by itself.

8. A method for treating a cancer in a subject in need thereof, the method comprising administering to the subject an effective amount of a composition comprising a targeted activated T cell which selectively binds a cell of the cancer in the subject; and administering an effective amount or a subtherapeutic amount of an additional therapeutic agent to the subject, to thereby treat the cancer in the subject.

9. The method of claim 8, wherein the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid or liquid tumor.

10. The method of claim 8 or claim 9, wherein the cancer is a drug resistant cancer or drug sensitive cancer.

11. The method of any one of claims 8-10, wherein the cancer is selected from the group consisting of pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma.

12. The method of any one of claims 8-11, wherein the effective amount or the subject therapeutic amount of the additional therapeutic agent is administered contemporaneously with or after the administering of the targeted activated T cell.

13. The method of any one of claims 8-12, wherein the additional therapeutic agent is a chemotherapeutic agent or an anti-neoplastic agent.

14. The method of any one of claims 8-13, wherein the subtherapeutic amount of the additional therapeutic agent is a lower amount as compared to an amount of the additional therapeutic agent administered to a subject when the additional therapeutic agent is administered to a subject by itself.

15. The method of any one of claims 9-14, wherein the BAT comprises anti-CD3 x anti-EGFR BiAb, anti-CD3 x anti-HER2 BiAb, anti-CD3 x anti-GD2 BiAb, anti-CD3 x anti-CD20 BiAb, or anti-CD3 x anti-SLAMF7 BiAb.

16. The method of any one of claims 9-15, wherein the BiAb used to arm the targeted activated T cell is a chemically heteroconjugated bispecific antibody or a recombinant bispecific antibody of any configuration.

17. The method of any one of the preceding claims, wherein the targeted activated T cells are produced from an apheresis product.

18. The method of claim 17, wherein the targeted activated T cells are produced from an apheresis product by anti-CD3 stimulation in the presence of IL-2, optionally at a range of about 20 to about 200 IU/ml, or wherein co-stimulated T cells are produced from an apheresis product by co-stimulation with anti-CD3/anti-CD28 coated beads, optionally in the presence of IL-2 at a range of about 20 to about 200 IU/ml, optionally at bead to cell ratios from about 1:3 to about 3:1.

19. The method of any one of the preceding claims, wherein the subject is a mammalian subject.

20. The method of any of the preceding claims, wherein the composition comprising the targeted activated T cell and/or the additional therapeutic agent is/are adapted for administration for the treatment of a subject by intravenous administration, intrathecal injection, peritoneal injection, or direct injection into the tumor or surrounding tumor site.

21. A pharmaceutical composition comprising, consisting essentially of, or consisting of an effective amount of a targeted activated T cell for use in treating a cancer in a subject in need thereof, wherein the targeted activated T cell selectively binds a cell of the cancer in the subject.

22. A pharmaceutical composition comprising, consisting essentially of, or consisting of an effective amount of a targeted activated T cell for use in a method for treating a cancer in a subject in need thereof, in combination with an effective amount or a subtherapeutic amount of an additional therapeutic agent, wherein the targeted activated T cell selectively binds a cell of the cancer in the subject.

23. The pharmaceutical composition of claim 21 or claim 22, wherein the targeted activated T-cell is selected from the group consisting of a bispecific antibody (BiAb) armed activated T cell (BAT), a tumor infiltrating lymphocyte, a CAR-T cell, and a bionic T cell engaging a cancer of hematologic origin or a solid or liquid tumor.

24. The pharmaceutical composition of any one of claims 21-23, wherein the cancer is a drug resistant cancer or drug sensitive cancer.

25. The pharmaceutical composition of any one of claims 21-24, wherein the cancer is selected from the group consisting of pancreatic cancer, breast cancer, prostate cancer, lung cancer, head and neck cancer, non-Hodgkin's lymphoma, acute myelogenous leukemia, acute lymphoblastic leukemia, neuroblastoma, and glioblastoma.

26. The pharmaceutical composition of any one of claims 22-25, wherein the effective amount or the subtherapeutic amount of the additional therapeutic agent is administered contemporaneously with or after the administering of the targeted activated T cell.

27. The pharmaceutical composition of any one of claims 22-26, wherein the additional therapeutic agent is a chemotherapeutic agent or an anti-neoplastic agent.

28. The pharmaceutical composition of any one of claims 22-27, wherein the subtherapeutic amount of the additional therapeutic agent is a lower amount as compared to an amount of the additional therapeutic agent administered to a subject when the additional therapeutic agent is administered to a subject by itself.

29. The pharmaceutical composition of any one of claims 23-28, wherein the BAT comprises anti-CD3 x anti-EGFR BiAb, anti-CD3 x anti-HER2 BiAb, anti-CD3 x anti-GD2 BiAb, anti-CD3 x anti-CD20 BiAb, or anti-CD3 x anti-SLAMF7 BiAb.

30. The pharmaceutical composition of any one of claims 23-29, wherein the BiAb used to arm the targeted activated T cell is a chemically heteroconjugated bispecific antibody or a recombinant bispecific antibody of any configuration.

31. The pharmaceutical composition of any one of claims 21-31, wherein the targeted activated T cells are produced from an apheresis product.

32. The pharmaceutical composition of claim 28, wherein the targeted activated T cells are produced from an apheresis product by anti-CD3 stimulation in the presence of IL-2, optionally at a range of about 20 to about 200 IU/ml, or wherein co-stimulated T cells are produced from an apheresis product by co-stimulation with anti-CD3/anti-CD28 coated beads, optionally in the presence of IL-2 at a range of about 20 to about 200 IU/ml, optionally at bead to cell ratios from about 1:3 to about 3:1.

33. The pharmaceutical composition of any one of claims 21-32, wherein the subject is a mammalian subject.

34. The pharmaceutical composition of any one of claims 22-33, wherein the composition comprising the targeted activated T cell and/or the additional therapeutic agent is/are adapted for administration for the treatment of a subject by intravenous administration, intrathecal injection, peritoneal injection, or direct injection into the tumor or surrounding tumor site.
